(19) United States
(12) Patent Application Publication
(10) Pub. No.: US 2008/0212734 A1
(43) Pub. Date: Sep. 4, 2008

(54) Correction of Non-Linearities in an Imaging System by Means of a Priori Knowledge in Radiography

(75) Inventors: Stefan Kasperl, Erlangen (DE); Ingo Bauscher, Igensdorf (DE); Matthias Franz, Fgrth (DE); Stefan Schroepfer, Oberasbach (DE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Fraunhofer-Gesellschaft zur Foerderung der angewandten Forschung e.V., Muenchen (DE)

(21) Appl. No.: 11/908,267

(22) PCT Filed: Mar. 9, 2006
(86) PCT No.: PCT/DE06/00420

§ 371 Date: Sep. 10, 2007

(30) Foreign Application Priority Data
Mar. 9, 2005 (DE) 10 2005 011 161.0

(51) Int. Cl. A61B 6/00; G01D 18/00

(52) U.S. Cl. 378/4; 378/207

(57) ABSTRACT

The invention relates to a method for online correction of non-linearities in the imaging system during the data acquisition in industrial computer tomography (CT). The above provides a method for the provision of corrected projection data as an improved CT reconstruction, whereby measuring beams (q) are emitted from a radiation source (Q) which pass through the sample (10,11), the intensity of which is recorded by a detector (31). The following steps are provided: a first initialization, whereby a first orientation of the sample (10) is merely coarsely determined with a first rapid recording, a recording in which the position of the sample (10) is more accurately determined, in particular by feature point pairs, a movement, whereby after a successful recording of several projections, the position of the sample (10,11) is calculated for at least one further projection, a simulation, whereby a virtual CT is carried out using the results from the previous step, providing input data for an ensuing correction method for the CT reconstruction, carrying out a correction, whereby during data recording (70) by the detector, parameters are determined from the correction data and a correction is then carried out (73a,73b) and the reconstruction, whereby in the period at the end of the recording process corrected projection data (11*) as a data recording (70) are provided as an improved CT reconstruction (74,75).

### CROSS-REFERENCE TO RELATED APPLICATIONS

This application is a U.S. national stage application of International Application No. PCT/DE2006/000420, filed Mar. 9, 2006, which claims the benefit of German Patent Application No. DE 10 2005 001 161.0, filed on Mar. 9, 2009, the disclosure of which is herein incorporated by reference in its entirety. PCT/DE2006/000420 designated the United States and was not published in English.

### FIELD OF INVENTION

The present invention relates to tomographic methods, and particularly to correction of non-linearities in computer tomography (CT).

### BACKGROUND INFORMATION

In computer tomography, different physical effects cause artifacts in the reconstructed tomograms, which decrease the image quality. Tomograms may be used in industrial quality control applications such as the quantitative measurement of objects. Decreasing the amount and severity of artifacts in computer tomography reconstructions (CT reconstructions) may allow for more precise measurements and enable measurement tasks to be automated.

CT systems generally operate in the following manner: A radiation source radiates through an object. The radiation passing through the object is weakened in its intensity depending on the length and absorption properties of the object in the optical path. A detector, which detects transmission values (i.e. intensity of the radiation that has passed through the object) is disposed behind the object. Typically, the detector is designed as a two-dimensional pixel detector, which provides a two-dimensional transmission picture of the object on the output side, wherein the intensity of the radiation passing through the object depends both on the absorption properties of the object, which can vary over the path of the radiation through the object, and on the transmission length of the object.

Typically, an X-ray radiation source is used as radiation source. As it is known, computer tomography works on the basis of transmission images. A computer tomographic image consists of a sequence of projections, wherein the object is first radiated through in a certain position, the transmission direction of the object is then altered (e.g. by 1 degree), and another projection is recorded. Thus, a computer tomographic image comprises a sequence of projections, wherein a rotation angle and general geometry data, respectively, are associated to every projection, wherefrom it can be derived how the position of the object has changed from one projection to the next. Additionally, every projection may include a two-dimensional array of transmission values, which are typically intensity values.

In an exemplary embodiment, 360° projections may be recorded, and the object may be rotated by 1 degree between two projections. Depending on the application, however, significantly more or significantly less projections are possible. The individual projections are processed with reconstruction methods (e.g. filtered reprojection) to generate three-dimensional volume data, which consists of a plurality of volume elements or voxels. In a three-dimensional computer tomography, a value may be associated to every voxel, which indicates the absorption density at a particular location.

Three-dimensional CT may be applied in the industrial quality control of devices under testing with regard to the quantitative measurement of objects. An exemplary application is the production of cast parts in the automobile industry. The quality control of cast parts comprises defect detection and dimension testing. Main tasks in the pre-series development are the fast checking of the dimensional stability of cast parts with complex geometry as well as the analysis of deviations of the geometry data from data contained in a part plan.

Under the aspect of industrial applicability in comparison to other sources (synchrotron or gamma radiator) X-ray tubes are preferably used as radiation sources. Instead of a line detector in the two-dimensional computer tomography, a flat X-ray detector is used in the three-dimensional computer tomography. The three-dimensional computer tomography requires only one rotation of the object for reconstruction, whereby measuring times are significantly reduced compared to two-dimensional computer tomography.

Conventional correction methods (e.g. beam hardening correction, or beam scatter correction) reduce artifacts and the image quality thus achieved allows useful dimension conformity analyses. These conventional methods, however, operate with an iterative sequence and require the availability of complete projection data. For example, a first CT-reconstruction may initially provide 3D voxel data with artifacts of the object. Post processing image processing steps may determine correction parameters therefrom for an improved second CT-reconstruction. If necessary, additional iterations may be performed. In CT-reconstructions with many artifacts, the input data required for the correction method may not be correctly determined from the object itself.

### SUMMARY

In an exemplary embodiment, a method is provided for online correction of non-linearities of an imaging system during data acquisition in industrial computer tomography (CT). The non-linearities of an imaging system may be corrected through the supplemental use of target date of an object image.

One application of one or more exemplary embodiments is a cast parts production in the automotive industry. Quality control of cast parts includes primarily finding voids and checking dimensions. An aspect of pre-series development is a quick check of the dimensional compliance of cast parts with complex geometry and the analysis of the deviations from the target data. In industrial applications, X-Ray Tubes, in comparison to other sources (synchrotron or gamma radiation emitter), may be used as radiation emitters.

The X-ray tubes used in CT, however, emit a polychromatic radiation. The interaction of the X-rays when passing through materials may be energy dependent. Characteristic curves of real systems thus have a non linear extension, caused by effects like beam hardening, beam scatter and non-linearities of the detector. This may cause artifacts in the reconstructed layers, like stripes, blurred edges, drum shaped distortions and cupping effects, degrading image quality and making measurement tasks difficult.

The method claimed herein corrects non-linearities of the imaging system computer tomography during data acquisition or at least calculates parameters used therein before the end of the data acquisition (acquisition process or abbreviated “data acquisition”). The image quality of the reconstruction is thus improved and quantitative measurements tasks may be accomplished, including the testing of dimensional compliance or target versus actual comparisons of the object body with target data (e.g. from a CAD system).

The claimed method operates with a single CT-reconstruction. Time consuming iterative post processing steps (JAR) may therefore be omitted. Through the use of the target data of the object as a priori knowledge, the correction methods can use better input data, which produces better quality CT-reconstructions. The method uses the target data of the object and delivers input data for correction methods of the CT-reconstruction.

An exemplary embodiment is a multi stage method, the single stages of which are;

Initialization: The orientation of the object is roughly determined through a first, fast recording.

Recording: Starting with the rough positioning, a recording is performed, based on features and/or intensities. This is a more precise recording.

Movement: After a successful recording in some projections, the position of the object can be computed, e.g. with respect to the rotation axis for further projections.

Simulation: Based on this knowledge a virtual CT can be simulated, delivering the required input data for the correction methods of the CT-reconstruction.

Correction: The correction parameters are determined during data acquisition. A correction is performed either now or later.

Reconstruction: At the end of the acquisition process corrected projection data for an improved CT-reconstruction of the object are available.

Initialization means a coarse grid recording of the object. A coarse grid recording thus is a recording, whose precision is a few degrees in rotation, in particular, above an angular error of approximately 1 degree: and/or approximately 1 mm to 2 mm with respect to translational movement, or in a range of 1% of a typical object dimension.

Thus a start value may be formed, which is being used for a more precise recording, performed subsequently. For this purpose, certain pairs of feature points may be used.

The precise recording may be performed based on features of the object and/or intensity “based” in the sense of an evaluation of this measurement data.

Feature Based Recording:

After a coarse recording (e.g. a determination of a coarse grid angular value of a rotatably supported object, possibly also with an associated linear movement), singular point pairs are being searched, wherein a singular point is a point which stands out from its environment in a measurable manner. These singular points on the one hand can be points which have a maximum or a minimum, two dimensional and also one dimensional. The singular point standing out from its environment is also measurable. Other possibilities for singular points that need to be understood are peripheral points of the object shadow, or intersection points of edges.

One point of a digital model of an object (mostly of a CAD model) is formed on the detector during projection. The singular point of the model and the singular point of the image form a point pair, which is designated as “feature point.”

If the model is recorded coarsely, projections can be simulated. Through these simulations approximate positions of projections of model feature points can be derived according to the coarse recording. These positions are known to the computation. Such knowledge, however, can also be initially acquired through the coarse recording of the CAD-model, which subsequently brings the simulation to the approximate position of the projection.

Feature points can also be extracted from the measurements. This extraction of said singular points (in the sense of preferably unique feature points) is performed through search algorithms from the measurements. The search algorithms are adapted to the simulated projection of the digital model.

Now, since there are feature points (as point pairs), the position can be recorded at the beginning of the CT-scan. This recording is performed from a projection. Possible usable algorithms to perform this recording include the process SoftPOSIT (see DeMenthon et al., Soft POSIT Simultaneous Pose and Correspondence Determination, International Journal of Computer Vision, 59 (3), 2004, pages 259-284). This possibility of recording the starting position is relatively insensitive towards erroneously associated feature point pairs, as long as they are not too numerous, when the process SoftPOSIT is applied.

Intensity Based Recording;

The procedure of intensity based recording is to determine the similarity between reference and template image. Herein similarities are derived through statistical methods, all pixel information is used as a reference, (see Penny et al., “A Comparison of Similarity Measures for Use in 2-D-3-D Medial Image Recording”, IEEE Transactions on Medical Imaging, 17(4), 1998, pages 586-595). Intensity based 2D or 3D recording algorithms optimize the similarity of reference and transformed template, based on a sufficiently good starting value, (see Pluim, IEEE Transactions on Medical Imaging, 22(8), pages 986-1004).

This way a priori knowledge can be used to successfully perform a recording. The CT model as target data of the object, and the a priori knowledge thereby applied, can be used at several projections in various positions of the object. Each position is characterized by another rotation angle, which is assumed by an object with reference to a rotation axis.

The recording as a 2D recording or 3D recording is performed alternatively and caused by the application. From a 2D fan beam CT, a generalization to a 3D cone beam CT can be performed without problem. The type and method of the detector is adapted accordingly, wherein said detector is either provided as a line detector in a 2D-CT, or as a surface detector in a 3D-CT. Under both assumptions reduced intensities are imaged onto the detector through the object and through the permeation of the object with the measurement radiation from the punctiform source, as a respective projection at a respective rotation angle of the object.

The ideal case is a perfectly aligned CT imaging system. In this case only the position of the rotation axis has to be known, around which the object is rotated in angular increments.

These angular increments between the receiving positions of the object are well known, so is the recording. With a recorded digital model of the object it is possible now to perform a CT simulation. This CT simulation can be performed for any detector pixel on the detector at any rotation position of the object yielding an associated irradiated length of an imaginary measurement beam originating from a punctiform source.

The recording at some projections allows using the CT at remaining projections, so that the length of the object can be computed for additional projections.

A simulation in the form of a virtual CT can be performed based on the above knowledge. It yields the necessary input data for correction methods during reconstruction.

A correction, at least a provision of correction parameters, may be performed during data acquisition. In a virtual CT, associated irradiated lengths are created for any detector location (pixel) at any assumed incremental rotation position of the object. A respective irradiated length and associated measured intensity at the detector may be combined into data pairs. In order to determine the correction data during data acquisition, data from all projections are not necessary.

A few projections are enough (e.g. a representative choice covering an angular area below 360°). Since the correction data are already determined during data acquisition, and not all projections are necessary as input variables in order to determine the correction parameters, the determination of the correction parameters can already be begun when this representative choice of projections is recorded. This way at least part of the computation of the correction parameters and the additional acquisition process run in parallel. The computation of the correction parameters can preferably be completed, or become complete substantially at the end of the acquisition process, thus also of those projections, which are not necessary for the representative choice. The reconstruction can be performed in a time frame after, or right at the completion of the acquisition, thus allowing a smaller delay until the results are available.

Such methods can be applied as correction methods (See “Quality Improvements for Cone-beam CT using Beam Hardening and Scattering Correction”, Third World Congress on Industrial Process Tomography, Banff, Canada, 2002, pages 90-95.) for the reconstruction. Corrected projection data already exists, so that the first reconstruction can already operate with correction data. A reconstruction can be based on measurement data, which may have already been corrected. Already the first reconstruction yields a completely corrected volume of the reconstructed object. An improved CT reconstruction is achieved.

The input data used for the correction are better, which yields a better quality CT reconstruction.

Advantages of these and other embodiments will become apparent from the following detailed description, which taken in conjunction with the accompanying drawings, describe by way of example—and not limitation—principles of various exemplary embodiments.

### BRIEF DESCRIPTION OF THE DRAWINGS

Purposes and advantages of exemplary embodiments will be apparent to those of ordinary skill in the art from the following detailed description in conjunction with the appended drawings in which like reference characters are used to indicate like elements, and in which:

FIG. 1 is a schematic side view of an imaging system with a symbolization of a radiography, caused by a radiation source Q, measurement beams q, an object 10 and a detector 31;

FIG. 1(a) is a top view of the arrangement of FIG. 1, from which the rotary table with its axis 100 can be derived. The two peripheral points of the object form the boundary beams of the fan of the measurement beams q for imaging an intensity distribution at the detector 31, which forms a layer for a level, but which can depict a volume of the object in the form of a flat x/y extension, also in case of a 3 dimensional CT, wherein the detector 31 is provided flat accordingly;

FIG. 2 illustrates the incremental change of the angular position of the object by a respective differential angle Δα;

FIG. 3 illustrates, not necessarily to scale, but in a symbolic manner and highly enlarged for clarity, the recording of an object 11, which is shown in full lines in its actual position 11, and which is shown in dashed lines in its imprecise coarsely determined position 11′. The differential angle is designated as recording error y. The beam source Q may be much further away from the object than shown by the symbolic distance z1, the object 11 may also be further away from the detector than shown by the distance z2 in a symbolic manner;

FIG. 3(a) is the intensity profile, or the associated intensity profile in x-direction (in FIG. 3 from the top to the bottom) with reference to a punctiform beam source with a fan shaped beam as measurement beams. From this substantial feature points become evident, whose positions are designated xa, xe and xf, and which belong among the peripheral points 11a, 11e and 11f of the object 11 from FIG. 3; and

FIG. 4 illustrates a process diagram for performing the reconstruction with partially parallel determination of correction parameters, so that the corrected measurement data of the first reconstruction can already reconstruct a completely corrected volume 11*.

Advantages of these and other embodiments will become apparent from the following detailed description, which taken in conjunction with the accompanying drawings, describe by way of example—and not limitation—principles of various exemplary embodiments.

### DETAILED DESCRIPTION OF EXEMPLARY EMBODIMENTS

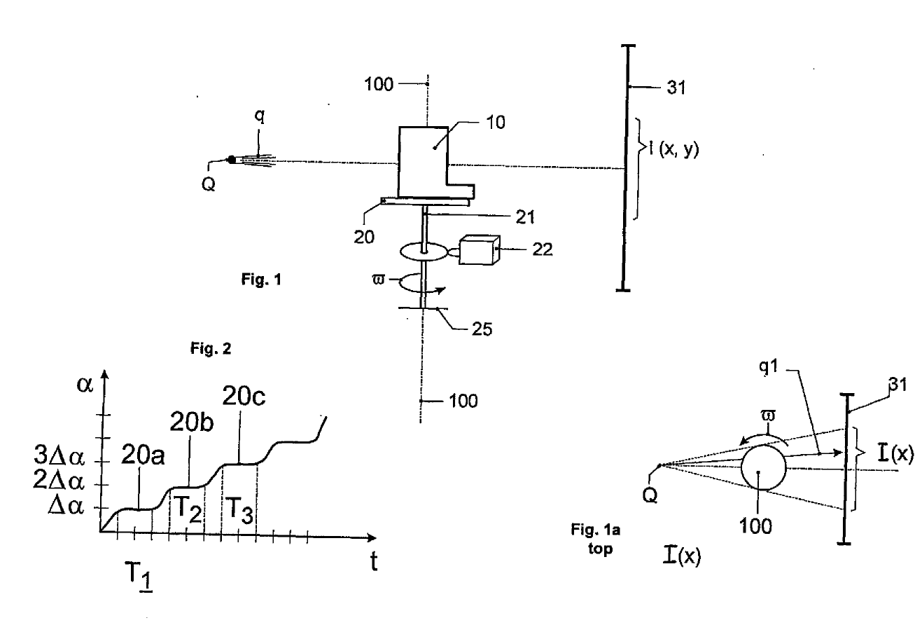

The side view of FIG. 1 shows an object 10 in L shape (in side view) and a radiation source Q which can deliver X-ray beams or neutron beams. These beams are designated with q, either cone shaped or fan shaped for a 2D- or a 3D tomography. The axis 100 is the rotation axis of a table 20 driving a shaft 21 through a drive 22 with a transmission, wherein the said shaft is coupled torque proof with the rotary table 20. The rotation is designated ω (omega). The shaft 21 is supported rotatably on a pedestal surface 25.

The axis 100 is perpendicular to the radiation axis extending from the source Q passing through the object 10 and to a screen 31, which is used as a detector. In the elevation direction of the illustration an intensity distribution I is shown, which has a 2 dimensional shape as I(x, y) in case of a 3 dimensional tomography with a reduced intensity distribution according to the shape, configuration and material of the object 10. In case of a radiation through a layer and a fan shaped beam q, for example, only an elevation direction is to be measured, having an intensity distribution I(y). This is the data of a radiography that needs to be acquired.

In a top view this assembly is shown as FIG. 1 a (without the object 10) with a rotary table 20 which can be rotated around the axis 100. The peripheral beams of the beam source Q are drawn barely touching the rotary table. also the beam axis, and also the intensity distribution I(x} in horizontal direction on the detector 31.

A drive beam q1 is illustrated which would radiate through the object 10 when put onto the rotary table 20 and which is located within the 2 object shade lines (boundary beams).

The rotary table 20 can be rotated by the drive 22 in steps by angular increments Δα, as illustrated by FIG. 2. A respective time span T1, T2, or T3 is an angular increment, which is valid for a radiography from the radiation source q. The angular increments are symbolized with 20a, 20b, and 20c in respective identical increments.

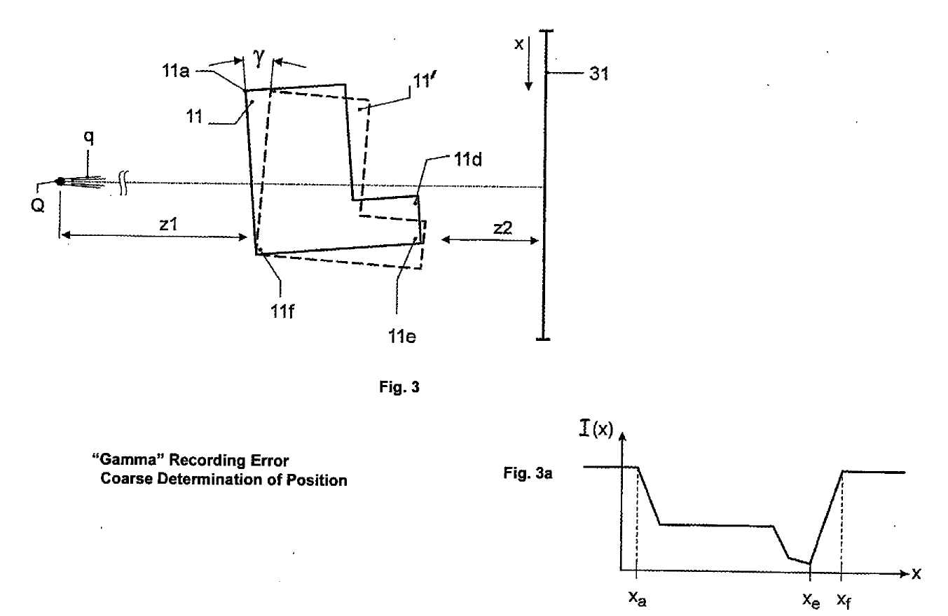

FIG. 3 illustrates the object in a symbolic manner, but not necessarily to scale, and with a similar shape to the object 11, designated in the coarse recording.

An orientation of the object 11 is coarsely determined in a first, fast recording. Thus, the object is located in the position which is drawn in bold lines, with the corner points 11a, 11e and 11f, and it is permeated by radiation from the radiation source Q (e.g. by the fan beam q). The beam axis is orthogonal to the detector plane 31 in case of a surface detector. In case of a line detector there is only a dependency from x. The position of the object 11 is defined precisely through feature point pairs. Other possibilities, which are described separately, are statistical methods, also achieving a positioning of the object, which is more precise than the first coarse (fast recording), identifying the coarse position 11′ of the object. In this case, an angular error y may be assumed, which is shown between the actual position 11 and the recorded position 11′. The angular error y may be more than one degree. In addition, a translatoric error can occur, which is located in the range above 1 mm to 2 mm, (or measured at the object as at least 1% of its largest, in particular typical length).

The distances z1, z2 are not necessarily drawn to scale, but they are symbolic.

The intensity distribution illustrated in FIG. 3a may occurs in case of a fan shaped beam q. The pattern of the fan shaped beams from the top to the bottom, starting with the corner point 11a to the corner point 11f (respective boundary beam) shows the pattern of FIG. 3a, according to the stronger increasing or decreasing thickness of the object 11 absorbing the radiation. The diagram of the intensity I(x) shows a few singular points at the positions xa, xe, and xf, corresponding to the corner points 11a, 11e, and 11f of the position of the object. In case of a respective imprecise recording, 11′, the function diagram of FIG. 3a moves in x-direction by a small amount.

Each singular point forms a point pair with a respective model point in a digital model, mostly a CAD model of the object. Several such point pairs can each accomplish a more precise recording of the object in a projection.

The measurements of the singular points on the detector can be understood as an extraction. By all means they make the positioning of the object more precise beyond the coarse recording. Alternatively, statistical methods can be applied as described above. The similarity between the reference image and the template image plays an important role herein, (see Penney in IEEE transactions, mentioned above). These statistical methods may operate on an intensity basis and may allow for a more precise recording.

When insofar a “sufficiently precise determination” is mentioned, this is certainly more precise than the fast coarse recording and the coarse determination of the position of the object, which served as a starting point.

After a successfully performed recording in at least some projections, the position of the object 11 relative to the rotation axis can be determined, possibly also with a translatory error for at least one additional projection.

The influence of the target data of the object from the digital model may allow for improvement of the coarse positioning of the object. After such a performed recording, at least one additional projection of the object can be computed. This can be performed in reference to the rotation axis and/or with a translatoric motion.

After a recording of the object, a virtual CT can be performed through the acquired knowledge. This is a simulated CT through which input data for a correction method are provided for the reconstruction. This is only possible when the coarse recording has been performed. A use of the correction data, which are generated by the simulation, can either already start while the data acquisition is being performed, or only after the completion of this data acquisition, in the time frame around the end of the acquisition process.

The necessary correction data, which has already been determined during the data acquisition, is available at the end of the acquisition process. Accordingly, a fast correction may already have correction parameters available for a reconstruction at the completion of the data acquisition. As a consequence, large time savings of the computation method occur.

From the correction data which were already determined during the data acquisition, the correction, and thus the reconstruction at the end of the acquisition process can provide an improved CT reconstruction. Already the first reconstruction can operate with correction data, which are available directly at the end of the acquisition process, after they were previously determined during the data acquisition.

A correction may also be performed during the acquisition process (the data acquisition). The correction may be performed on a portion of the artifacts, which are generated during the data recording. A reconstruction of the measured object may thus be performed with corrected measurement data that is available more quickly and is also of a better quality.

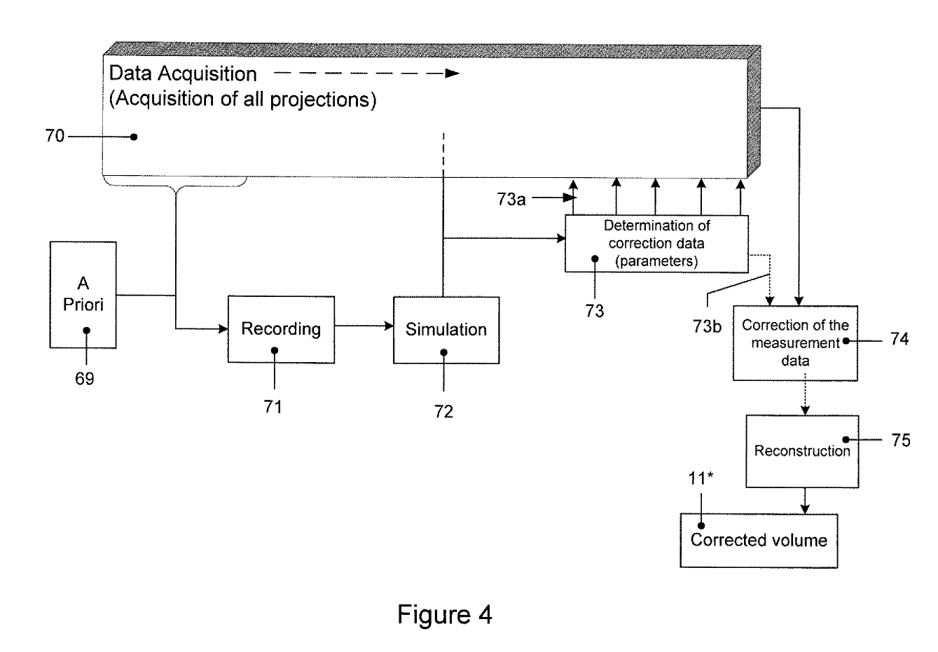

FIG. 4 illustrates a symbolic signal flow pattern, or schedule of a data acquisition 70, which can be viewed time based, starting on the left with its beginning and with its end on the right. A priori knowledge 69 is initially predetermined and allows a recording 71, which is coarse and which can be provided more precisely through the use of e.g. feature point pairs, which are respective singular measurable point(s) on the detector 31, and which are paired with respective associated singular point(s) in the digital model. The successful recording then allows a simulation 72, which is a virtual CT. Input data for correction methods of the CT reconstruction are delivered by it.

During the determination of the correction data 73, which is already performed during the data acquisition 70, correction data are determined which can lead to a correction of the data of the data acquisition 70, which is symbolized by the arrows 73a. Thus, a correction 73b can be performed subsequently in an alternative embodiment, or also cumulative, when the data acquisition is complete, and the projection or data acquisition is handed over to the computations “correction of the measurement data” 74. From this correction, which can be performed very quickly time wise, a reconstruction 75 is generated, which can also be performed very quickly, in order to obtain the corrected volume 11*, which forms the reconstruction.

At the end of the acquisition process, the right edge of the block “acquisition” 70 symbolizes the section before the immediate end through the influence of the correction parameters by the influences 73a onto the data acquisition, and/or the section 74, 73b, which is positioned subsequently, and which relates to the correction and the reconstruction.

The industrial quality control is an exemplary area of application, in particular in the area of automotive construction, and with reference to the cast parts as objects 10, 11. X-ray beams are mentioned as exemplary measurement beams.

Through the setup according to FIG. 4, the artifacts can also be reduced without iteration, and this can be performed with large time savings. The projections used in the parameter determination are fewer than all images made available for a rotation angle of 360°, which are acquired in increments Δα.

1. A multi stage method for providing corrected projection data as an improved CT reconstruction, the method comprising:
providing a first projection by emitting beams from an emission source, wherein the beams pays through an object onto a detector configured to detect and record the intensity of the beams:
coarsely recording the detected beams to determine an approximate position of the object, wherein the coarse recording is used for extracting unambiguous feature points;
determining the position of the object, with sufficient precision, by using pairs of feature points;
rotating the object about an axis in predetermined angular increments;
providing one or more further projections of the object onto the detector after each of one or more rotations;
computing the position of the object for each of the one or more further projections; performing a simulation in the form of a virtual CT, based on the computed positions of the object, to yield simulation data, wherein simulation data is input data for a correction method of the CT reconstruction;
determining correction parameters from the simulation data during the data acquisition process, and using the correction parameters to correct projection data;
performing CT reconstruction on the object based on corrected projection data, wherein the correction parameters for reconstruction are available at the completion of data acquisition; and
wherein a 2D or 3D recording with reference to target data of the sample is performed with the measured data, based on the extracted feature points.

2. The method of claim 1, wherein the CT reconstruction is performed in the context of industrial quality control.

3. The method of claim 2, wherein at least one measurement is performed on the object.

4. The method of claim 1, wherein x-rays are used for performing the CT.

5. The method of claim 1 or 3, wherein the object is a cast part in automotive construction.

6. The method of claim 1, wherein no iteration is used in the CT reconstruction.

7. The method of claim 1, wherein the input data for the correction method are data pairs, which are comprised of the respective irradiated length and the associated measured intensity on the detector.

8. The method according to claim 7, wherein the object rotations are substantially less than 360°.

9. The method according to claim 1, wherein the feature points are a respective singular point pair, comprised of a model point and associated point of the projection.

10. A multi stage method for providing corrected projection data as an improved CT reconstruction, in which fan shaped measurement beams are emitted by a beam source, said measurement beams irradiating through the sample, and their intensity being detected on a detector, the method comprising:
providing a first projection by emitting measurement beams from an emission source, wherein the measurement beams pass through an object onto a detector configured to detect and record the intensity of the measurement beams;
coarsely recording the detected beams to determine an approximate position of the object;
determining the position of the object, with sufficient precision, by using pairs of feature points;
rotating the object about an axis in predetermined annular increments;
providing one or more further projections of the object onto the detector after each of one or more rotations;
computing the position of the object for each of the one or more further projections;
performing a simulation in the form of a virtual CT, based on the computed positions of the object, to yield simulation data, wherein simulation data is input data for a correction method of the CT reconstruction;
determining correction parameters from the simulation data during the data acquisition process, and using the correction parameters to correct projection data; and
performing CT reconstruction on the object based on corrected projection data, wherein the correction parameters for reconstruction are available at the completion of data acquisition.

11. The method of claim 10, wherein non-linearities of the imaging system, comprised of source and detector are corrected with an object put between the source and detector.

12. (canceled)

13. (canceled)

14. The method of claim 10, wherein x-ray beams are used as measuring beams in the process of a tomogram generation as a reconstruction of the object.

15. (canceled)

16. The method of claim 10, wherein the initialization is performed with an angular error of few degrees, in particular above 1°, and/or with a translatoric error above substantially 1 mm.

17. The method of claim 16, wherein the rotation axis of the object is given, around which the sample is rotated in single indexed steps of predetermined angular increments Δα during radiography.

18. The method of claim 10, wherein the feature points are extracted, and thus a respective singular point from a digital model, in particular a CAD model, appears on the detector as a respective imaged point, and both corresponding points form a feature point pair.

19. A method according to claim 10 or 16, wherein the initialization is performed with a translatoric error, substantially in the range of 1% of a typical dimension of the sample.

20. A multi stage method for providing corrected projection data as an improved CT reconstruction, in which fan shaped measurement beams are emitted by a beam source, said measurement beams irradiating through the sample, and their intensity being detected on a detector, the method comprising:
providing a first projection by emitting measurement beams from an emission source, wherein the measurement beams pass through an object onto a detector configured to detect and record the intensity of the measurement beams;
coarsely recording the detected beams to determine an approximate position of the object;
determining the position of the object with sufficient precision;
rotating the object about an axis in predetermined angular increments;
providing one or more further projections of the object onto the detector after each of one or more rotations;
computing the position of the object for each of the one or more further projections;
performing a simulation in the form of a virtual CT, based on the computed positions of the object, to yield simulation data wherein simulation data is input data for a correction method of the CT reconstruction;
determining correction parameters from the simulation data during the data acquisition process, and using the correction parameters to correct projection data; and
performing CT reconstruction on the object based on corrected projection data, wherein the correction parameters for reconstruction are available at the completion of data acquisition.

21. The method of claim 20, wherein the positioning of the sample is performed through feature point pairs.

22. The method of claim 20 or 21, wherein the positioning of the sample is performed through an intensity based statistical method.
